(19) United States
(12) Patent Application Publication
(10) Pub. No.: US 2009/0171167 A1
(43) Pub. Date: Jul. 2, 2009

(54) System And Method For Monitor Alarm Management

(75) Inventor: Clark R. Baker, JR., Newman, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/342,956

(22) Filed: Dec. 23, 2008

Related U.S. Application Data
(60) Provisional application No. 61/009,230, filed on Dec. 27, 2007.

(51) Int. Cl. A61B 5/00

(52) U.S. Cl. 600/301

(57) ABSTRACT

Embodiments disclosed herein may include a method and system for determining patient-specific alarm thresholds for monitoring the patient's physiological parameters. For example, in an embodiment, the patient's age, weight, height, diagnosis, medications and/or other factors may affect his or her normal heart rate, blood oxygen saturation, and/or other physiological parameters. Accordingly, in an embodiment, information specific to or generally applicable to the patient may be supplied to a monitoring system to enable determination of the appropriate maximum and minimum thresholds. In an embodiment, if the patient exceeds one of the personalized thresholds, the monitoring system may alert a caregiver that there is a problem.

### RELATED APPLICATIONS

This application claims priority to U.S. Provisional Application No. 61/009,230, filed Dec. 27, 2007, and is incorporated herein by reference in its entirety.

### BACKGROUND

1. Field

The present disclosure relates to alarms in medical diagnostics apparatus and, in particular, to improvements in alarm limits based on various patient criteria.

2. Background

This section is intended to introduce the reader to various aspects of art that may be related to various aspects of the present disclosed embodiments, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of healthcare, caregivers (e.g., doctors and other healthcare professionals) often desire to monitor certain physiological characteristics of their patients. Accordingly, a wide variety of monitoring devices have been developed for monitoring many such physiological characteristics. These monitoring devices often provide doctors and other healthcare personnel with information that facilitates provision of the best possible healthcare for their patients. As a result, such monitoring devices have become a perennial feature of modern medicine.

One technique for monitoring physiological characteristics of a patient is commonly referred to as pulse oximetry, and the devices built based upon pulse oximetry techniques are commonly referred to as pulse oximeters. Pulse oximeters may be used to measure and monitor various blood flow characteristics of a patient. For example, a pulse oximeter may be utilized to monitor the blood oxygen saturation of hemoglobin in arterial blood (SpO₂), the volume of individual blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

In addition to monitoring a patient's physiological characteristics, a pulse oximeter or other patient monitor may alert a caregiver when certain physiological conditions are recognized. For example, a range of normal operation for a particular physiological parameter of a patient may be defined by setting low and high threshold values for the physiological parameter, and an alarm may be generated by the monitor when a detected value of the physiological parameter is outside the normal range. When activated, the alarm may alert the caregiver to a problem associated with the physiological parameter being outside of the normal range. The alert may include, for example, an audible and/or visible alarm on the oximeter or an audible and/or visible again at a remote location, such as a nurse station. These patient monitors may generally be provided with default alarm thresholds. However, in some instances, the default thresholds are not optimal for a given patient. For example, it may be desirable to have a higher or lower threshold based on the patient's age, weight, height, diagnosis, medications and/or other factors.

### SUMMARY

Certain aspects commensurate in scope with this disclosure are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

According to an embodiment, there may be provided a method for setting alarm thresholds, including receiving patient characteristics onto a memory device and using a processor to determine alarm thresholds based on the patient characteristics.

According to an embodiment, there may be further provided a monitoring system, including a monitor configured to calculate a physiological parameter of a patient and a processor configured to determine an alarm threshold for the physiological parameter based on at least one characteristic of the patient.

According to an embodiment, there may be still further provided a patient monitoring system, including an input device for receiving information about a patient, a first memory device for storing specifications for determining alarm thresholds based on the received information, a processor for determining the alarm thresholds based on the received information and using the stored specifications, and a second memory device for storing the determined alarm thresholds for comparison to physiological parameters of the patient.

Additionally, according to an embodiment, there may be provided a method, including receiving signals corresponding to absorption of light in a patient's tissue from a sensor, calculating the patient's physiological parameters based on the signals, receiving patient characteristics, determining threshold ranges of the physiological parameters based on the patient characteristics, and providing a notification if at least one of the calculated physiological parameters is outside a corresponding threshold range.

Finally, according to an embodiment, there may be provided tangible, machine readable media, comprising code executable to calculate a physiological parameter based on signals received from a sensor and determine an alarm threshold for the physiological parameter based on patient characteristics received from an input device.

### BRIEF DESCRIPTION OF THE DRAWINGS

Advantages of this disclosure may become apparent upon reading the following detailed description and upon reference to the drawings in which:

FIG. 1 is a perspective view of a pulse oximeter coupled to a multi-parameter patient monitor and a sensor in accordance with aspects of an embodiment;

FIG. 2 is a block diagram of the pulse oximeter and sensor coupled to a patient in accordance with aspects of an embodiment; and

FIG. 3 is a flow chart of a process related to determination of safe thresholds for the patient's physiological parameters in accordance with aspects of an embodiment.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Different patients may exhibit different normal ranges of physiological characteristic values. Factors such as age, weight, height, diagnosis, and a patient's use of certain medications may affect the patient's normal ranges of physiological parameters. For example, with a neonate, the normal SpO₂ range may be 80-95 percent, a n d the normal pulse rate range may be 90-190 beats per minute. In contrast, for a 40-year-old patient) the normal SpO₂ range may be 85-100 percent, and the normal pulse rate range may be 40-160 beats per minute. Accordingly, it may be desirable to set different low and high thresholds for particular parameters based on the patient being monitored. It should be noted that some patient characteristics may have more significance than others with respect to what may be considered a normal range for a particular physiological parameter. For example, the gestational age and weight of a neonate may significantly impact the normal ranges for SpO₂ and pulse rate.

Embodiments may be directed to a system and method for determining and setting alarm thresholds for certain physiological parameters of a patient based on patient characteristics. The patient characteristics may include, for example, the patient's age, weight, height, gestational age, diagnosis, and so forth. Additionally, patient characteristics may include medications taken by the patient, treatments the patient has received, and so forth. Furthermore, the patient characteristics may be approximated or selected from a list of possible values or ranges. For example, the patient's age may be specified by a range of ages, such as neonate, child 2-7 years old, child 8-12 years old, young adult 13-17 years old, adult 18-25 years old, adult 25-45 years old, etc. It is now recognized that such patient characteristics may affect the normal physiological parameters for the patient. Accordingly, low and/or high alarm thresholds for normal ranges of operation may be determined based on the patient's characteristics.

In accordance with present embodiments, rather than a caregiver determining these ranges and adjusting a monitoring device accordingly, the alarm threshold ranges may be determined from the patient characteristics input into the monitor. For example, monitors in accordance with present embodiments may be configured to determine the alarm threshold ranges for a given patient based on that patient's characteristics. The patient characteristics may be, for example, input by the caregiver, selected from a list of possible values or ranges, retrieved from a storage feature of a sensor or radio-frequency identification tag being used for the patient, or queried from a networked database including information about the patient. More specifically, a monitor component (e.g., a monitor memory) may receive input regarding patient characteristics directly from a caregiver (e.g., via a keyboard) or from a memory component of a device (e.g., a sensor) associated with a patient of interest. Based on the patient characteristics, the monitor may calculate or look up normal ranges for the particular patient's pulse rate and SpO₂, among other physiological parameters. Once identified, the thresholds appropriate for the patient may then be compared to the patient's measured physiological parameters. Such a comparison may facilitate identification of potential problems associated with the patient's actual physiological conditions having measured values outside of the identified normal range.

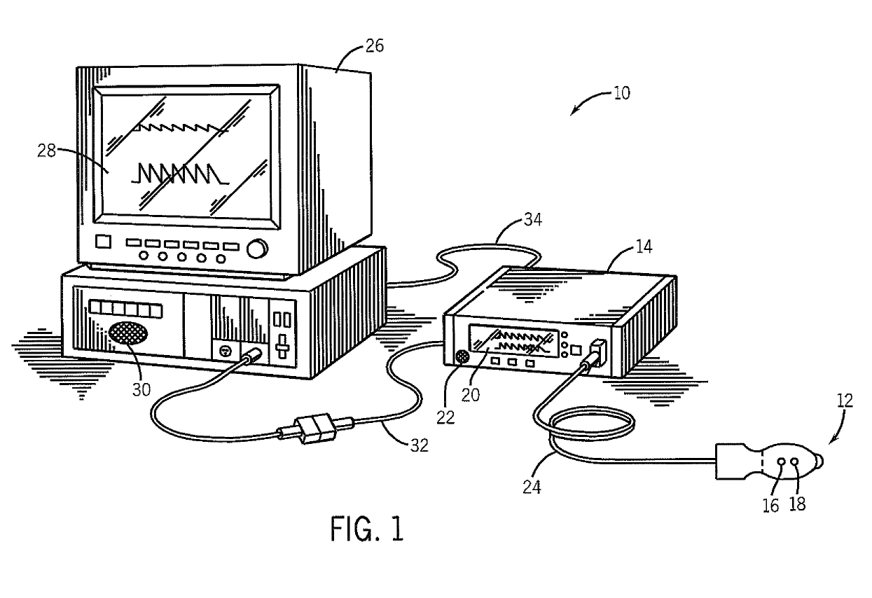

Although the techniques introduced above and discussed in detail below may be implemented for a variety of medical devices, the present disclosure will discuss the implementation of these techniques in a pulse oximetry system. FIG. 1 is a perspective view of such a pulse oximetry system 10 in accordance with an embodiment. The system 10 includes a sensor 12 and a pulse oximetry monitor 14. The sensor 12 includes an emitter 16 for emitting light at certain wavelengths into a patient's tissue and a detector 18 for detecting the light after it is reflected and/or absorbed by the patient's tissue. The monitor 14 may be configured to calculate physiological parameters received from the sensor 12 relating to light emission and detection. Further, the monitor 14 includes a display 20 configured to display the physiological parameters, other information about the system, and/or alarm indications. The monitor 14 also includes a speaker 22 to provide an audible alarm in the event that the patient's physiological parameters are not within a normal range, as defined based on patient characteristics. The sensor 12 is communicatively coupled to the monitor 14 via a cable 24. However, in other embodiments a wireless transmission device (not shown) or the like may be utilized instead of or in addition to the cable 24.

In the illustrated embodiment, the pulse oximetry system 10 also includes a multi-parameter patient monitor 26. In addition to the monitor 14, or alternatively, the multi-parameter patient monitor 26 may be configured to calculate physiological parameters and to provide a central display 28 for information from the monitor 14 and from other medical monitoring devices or systems (not shown). For example, the multi-parameter patient monitor 26 may be configured to display a patient's SpO₂ and pulse rate information from the monitor 14 and blood pressure from a blood pressure monitor (not shown) on the display 28. Additionally, the multi-parameter patient monitor 26 may emit a visible or audible alarm via the display 28 or a speaker 30, respectively, if the patient's physiological characteristics are found to be outside of the normal range. The monitor 14 may be communicatively coupled to the multi-parameter patient monitor 26 via a cable 32 or 34 coupled to a sensor input port or a digital communications port, respectively. In addition, the monitor 14 and/or the multi-parameter patient monitor 26 may be connected to a network to enable the sharing of information with servers or other workstations (not shown).

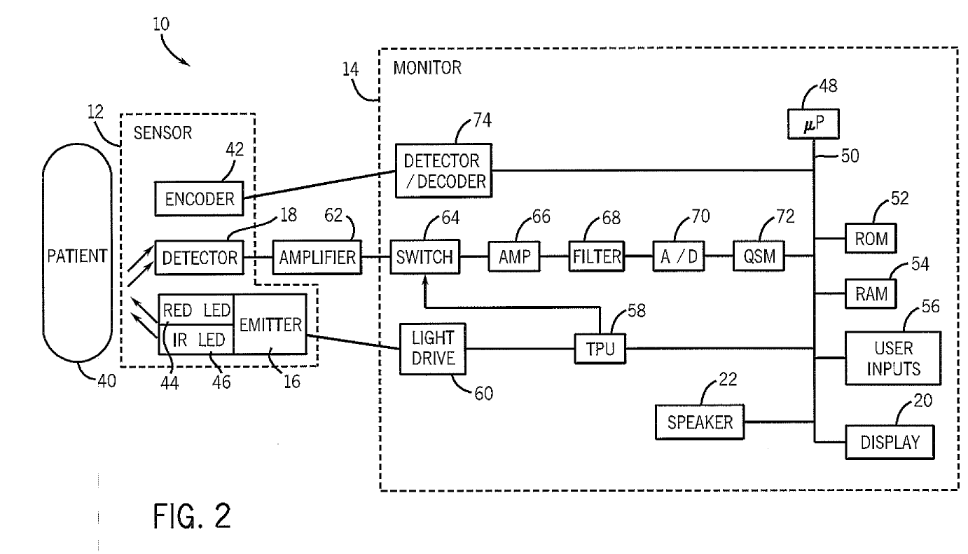

FIG. 2 is a block diagram of the exemplary pulse oximetry system 10 of FIG. 1 coupled to a patient 40 in accordance with present embodiments. One such pulse oximeter that may be used in the implementation of the present technique is the Model N600x available from Nellcor Puritan Bennett LLC, but the following discussion may be applied to other pulse oximeters and medical devices. Specifically, certain components of the sensor 12 and the monitor 14 are illustrated in FIG. 2. The sensor 12 includes the emitter 16, the detector 18, and an encoder 42. It should be noted that the emitter 16 is configured to emit at least two wavelengths of light, e.g., RED and IR, into a patient's tissue 40. Hence, the emitter 16 may include a RED LED 44 and an IR LED 46 for emitting light into the patient's tissue 40 at the wavelengths used to calculate the patient's physiological parameters. In certain embodiments, the RED wavelength may be between about 600 nm and about 700 nm, and the IR wavelength may be between about 800 nm and about 1000 nm. Alternative light sources may be used in other embodiments. For example, a single wide-spectrum light source may be used, and the detector 18 may be configured to detect light only at certain wavelengths. In another example, the detector 18 may detect a wide spectrum of wavelengths of light, and the monitor 14 may process only those wavelengths which are of interest. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present techniques.

In one embodiment, the detector 18 may be configured to detect the intensity of light at the RED and IR wavelengths. In operation, light enters the detector 18 after passing through the patient's tissue 40. The detector 18 converts the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, the detector 18 sends the signal to the monitor 14, where physiological parameters may be calculated based on the absorption of the RED and IR wavelengths in the patient's tissue 40. An exemplary device configured to perform such calculations is the Model N600x pulse oximeter available from Nellcor Puritan Bennett LLC.

The encoder 42 may contain information about the sensor 12, such as what type of sensor it is (e.g., whether the sensor is intended for placement on a forehead or digit) and the wavelengths of light emitted by the emitter 16. This information may allow the monitor 14 to select appropriate algorithms and/or calibration coefficients for calculating the patient's physiological parameters. In addition, the encoder 42 may contain information specific to the patient 40, such as, for example, the patient's age, weight, and diagnosis. This information may allow the monitor 14 to determine patient-specific threshold ranges in which the patient's physiological parameter measurements should fall and to enable or disable additional physiological parameter algorithms. The encoder 42 may, for instance, be a coded resistor which stores values corresponding to the type of the sensor 12, the wavelengths of light emitted by the emitter 16, and/or the patient's characteristics. These coded values may be communicated to the monitor 14, which determines how to calculate the patient's physiological parameters and alarm threshold ranges. In another embodiment, the encoder 42 may be a memory on which one or more of the following information may be stored for communication to the monitor 14: the type of the sensor 12; the wavelengths of light emitted by the emitter 16; the proper calibration coefficients and/or algorithms to be used for calculating the patient's physiological parameters and/or alarm threshold values; the patient characteristics to be used for calculating the alarm threshold values; and the patient-specific threshold values to be used for monitoring the physiological parameters.

Signals from the detector 18 and the encoder 42 may be transmitted to the monitor 14. The monitor 14 generally includes a microprocessor 48 connected to an internal bus 50. Also connected to the bus are a read-only memory (ROM) 52, a random access memory (RAM) 54, user inputs 56, the display 20, and the speaker 22. A time processing unit (TPU) 58 provides timing control signals to a light drive circuitry 60 which controls when the emitter 16 is illuminated and the multiplexed timing for the RED LED 44 and the JR LED 46. The TPU 58 also controls the gating-in of signals from detector 18 through an amplifier 62 and a switching circuit 64. These signals are sampled at the proper time, depending upon which light source is illuminated. The received signal from the detector 18 may be passed through an amplifier 66, a low pass filter 68, and an analog-to-digital converter 70. The digital data may then be stored in a queued serial module (QSM) 72 for later downloading to the RAM 54 as the QSM 72 fills up. In one embodiment, there may be multiple separate parallel paths having the amplifier 66, the filter 68, and the AID converter 70 for multiple light wavelengths or spectra received.

The microprocessor 48 may determine the patient's physiological parameters, such as SpO₂ and pulse rate, using various algorithms and/or look-up tables based on the value of the received signals corresponding to the light received by the detector 18. Signals corresponding to information about the patient 40 may be transmitted from the encoder 42 to a decoder 74. These signals may include, for example, encoded information relating to patient characteristics. The decoder 74 may translate these signals to enable the microprocessor to determine the thresholds based on algorithms or look-up tables stored in the ROM 52. In addition, or alternatively, the encoder 42 may contain the algorithms or look-up tables for identifying patient-specific alarm thresholds. The encoder 42 may also contain the patient-specific alarm thresholds, for example, if the alarm values are determined on a workstation separate from the monitor 14. The user inputs 56 may also be used to enter information about the patient, such as age, weight, height, diagnosis, medications, treatments, and so forth. In certain embodiments, the display 20 may exhibit a list of values which may generally apply to the patient, such as, for example, age ranges or medication families, which the user may select using the user inputs 56. The microprocessor 48 may then determine the proper thresholds using the user input data and algorithms stored in the ROM 52. The patient-specific thresholds may be stored on the RAM 54 for comparison to measured physiological characteristics.

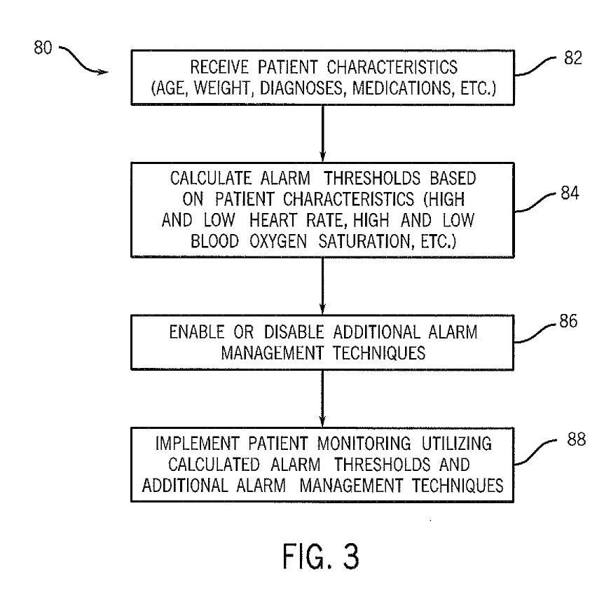

FIG. 3 is a flow chart illustrating an exemplary process 80 by which alarm thresholds may be set in a monitoring device, such as the pulse oximetry system of FIG. 1. A processing system may receive patient characteristics (block 82), such as age, gestational age, weight, diagnoses, medications, and so forth. The patient characteristics may be input by the caregiver, stored in the sensor, stored on a radio frequency identification tag associated with the patient, queried from a central database, or otherwise conveyed to the patient monitor. Based on the gathered data, patient-specific alarm thresholds for physiological parameters may be determined by the processing system (block 84). Determining the patient-specific alarm thresholds may include calculating the thresholds using algorithms or looking up the thresholds in one or more lookup tables. The processing system may include the monitor 14 (FIG. 1), the multi-parameter monitor 26, or another system. For example, the alarm thresholds may be calculated at a patient check-in workstation and transmitted to the monitor 14 via a network. In addition, the alarm thresholds may be calculated on one workstation and programmed into the RFID tag or sensor associated with the patient then transferred to the monitor 14 at a later time. Furthermore, additional alarm management techniques, such as integrated alarm thresholds and ventilation instability detection, may be enabled, disabled, or altered based on the patient characteristics (block 86) Monitoring of the patient's physiological parameters may then be implemented using the patient-specific alarm thresholds and/or alarm management techniques (block 88).

Specific examples of alarm thresholds which may be applied or modified based on patient characteristics may include, for example, high and low heart rate thresholds and high and low oxygen saturation thresholds. For example, the high heart rate threshold may be defined as a percentage of the maximum normal heart rate for the patient, while the low heart rate threshold may be based on the patient's minimum heart rate. Settings for the heart rate thresholds may be determined based on the patient's age. For example, a maximum adult heart rate may be calculated utilizing one the following equations:

HRₘₐₓ=220−Age, (1)

HRₘₐₓ=206−0.7(Age). (2)

A high heart rate threshold may be calculated as 80-90 percent of this age-specific value. A default low heart rate threshold may be 40-60 beats per minute for an adult, depending on the patient's age and overall physical condition. That is, a physically active young adult may have a lower minimum heart rate than an overweight or senior patient. Body mass index (BMI) may indicate whether a patient is overweight or obese. For example, BMI may be calculated using the following equation:

BMI=Weight(kg)/Height(m)². (3)

Accordingly, present embodiments may automatically set such heart rate thresholds based on patient characteristic data indicative of the patient's age and/or the patient's height and weight (i.e., BMI). In certain embodiments, the patient's characteristics may be approximated by a range, such as an age range, High and low alarm thresholds may then be determined based on the high range value, the low range value, the median range value, and so forth.

Similarly, the heart rate thresholds for neonates and children may be directly related to the patient's age or weight. For example, neonates have normal heart rates around 130 beats per minute, increasing to about 150 beats per minutes around 3 months of age, and gradually decreasing to about 70 beats per minute as a young adult. Accordingly, the heart rate thresholds for neonates may be adjusted based on the patient's age or weight, which is correlative of age. For example, a baby under 3 months of age or 5 kilograms may have a low heart rate threshold of 90 beats per minute, while an 18-year-old or a patient who weighs more than 60 kilograms may have a low heart rate threshold of 40 beats per minute. The high heart rate thresholds for neonates and children may also be adjusted according to the patient's age and/or weight.

In addition to age, height, and weight factors, the heart rate thresholds may be influenced by conditions that limit how fast or slow the individual patient's heart may beat. Conditions with which the patient has been diagnosed may be entered into the monitor as described above. A look-up table in the monitor may contain information on how the patient's condition affects heart rate thresholds. These conditions may include, for example, coronary artery disease (CAD), congestive heart failure (CHF), a history of arrhythmia, or recovery from open-heart surgery such as coronary artery bypass graft (CABG). For example, an adult patient prone to or at risk of having arrhythmias, such as ventricular tachycardia which may degenerate into ventricular fibrillation, may be in danger with a heart rate over 120 beats per minute. Accordingly, the high heart rate threshold for such a patient may be set at 120 beats per minute or less. The low heart rate threshold may also be adjusted if the patient is at risk for arrhythmias. For example, if the patient is prone to bradycardia, the low heart rate threshold may be increased to 50 beats per minute or higher. In addition, serious respiratory diseases may increase the risk of ventilation-perfusion mismatch during high blood flow, resulting in reduced arterial oxygenation at the highest heart rates, and therefore reduced oxygen availability to the heart to sustain those heart rates. Accordingly, a diagnosis of respiratory disease, such as pneumonia, chronic obstructive pulmonary disease (COPD), asthma, or acute respiratory distress syndrome (ARDS), may result in a reduced high heart rate threshold.

Furthermore, some medications may tend to decrease or increase heart rates. For example, medications known as negative chronotropes, such as beta-blockers, calcium channel blockers, acetylcholine, or digoxin, may slow a patient's heart rate due to their effects on electrical conduction, repolarization, or muscle contractions. A patient on any of these medications may therefore be expected to exhibit a lower high heart rate limit, and the threshold may be adjusted accordingly. Conversely, for a patient on anti-arrhythmic medications, known as positive chronotropes, the low heart rate threshold may be increased to 50 beats per minute or higher. Medications exhibiting positive chronotropy may include, for example, atropine, catecholamines such as epinephrine, or beta agonists such as dobutamine. As with the other patient characteristics, the patient's medications may be entered by the caregiver, selected from a list of possible medications or medication families, retrieved from a storage feature of a sensor or RFID tag, or queried from a networked database. The effect a given medication or family of medications typically has on a patient's physiological parameters may be, for example, stored in a look-up table, thereby enabling the monitor to make the appropriate threshold adjustments based on the patient's medications.

Additionally, a composite score representing the patient's overall disease state may be utilized to determine the patient's heart rate thresholds. For example, a metric such as the Acute Physiology and Chronic Health Evaluation (APACHE) II, Simplified Acute Physiology Score (SAPS) II or III, Paediatric Index of Mortality (PIM2), or another metric may be considered in setting the patient's high heart rate threshold. For example, a patient with an APACHE II score above 15 may have a narrower heart rate threshold range due to an increased low heart rate threshold and/or a decreased high heart rate threshold.

Similar to the high and low heart rate thresholds discussed above, high and low saturation thresholds may be set. As discussed below, a high saturation alarm threshold for a neonate may be set based on a gestational age or weight, while the threshold for an adult patient may be set based on diagnosis. Generally, for adults, a default high blood oxygen saturation limit may be 100 percent. However, for neonates, the default high saturation limit may be 95 percent as they are at risk of retinopathy of prematurity (ROP) if they receive too much oxygen or have unstable oxygenation. In very low birth-weight children, such as premature babies, it may be desirable to lower the high saturation limit even more. Accordingly, the high saturation threshold may be linked to a neonate's gestational age or weight. For example, the threshold may be 93 percent for a baby born at 26 weeks gestational age or weighing less than 1000 grams. The threshold may increase as the neonate ages and gains weight, for example, rising to 97 percent at 40 weeks gestational age or 4000 grams weight and to 100 percent at 52 weeks gestational age or 6000 grams weight.

Adult saturation levels are typically not influenced by age or weight, but they may be influenced by certain medical conditions, medications, or medical treatments. For example, adult patients diagnosed with chronic obstructive pulmonary disease (COPD) may live with and adapt to substantially increased carbon dioxide levels in the blood. These patients may be highly dependent on the hypoxic respiratory drive to maintain their spontaneous breathing, and may therefore require lower oxygen saturation levels. That is, a patient whose breathing relies on the hypoxic drive may decrease respiration upon an increase in oxygen saturation levels. Accordingly, the high saturation threshold for such patients may be decreased to as low as 90 percent, for instance, and the low saturation threshold may be decreased below 85 percent.

In addition, patients diagnosed with certain disorders or diseases may be more or less at risk of desaturation; therefore, the low saturation threshold may depend on the patient's diagnosis. For example, a patient with hypopnea or apnea may tend to experience frequent desaturation events which are expected and, therefore, do not necessitate an alarm. Accordingly, the low saturation threshold for such an adult patient may be set as low as 85 percent. Conversely, a patient with certain diagnoses or treatments may be at an increased risk upon a desaturation event. For example, a patient receiving supplemental oxygen, including mechanical ventilation, should be less prone to experiencing a desaturation event. Accordingly, when an event does occur, it is likely to be an indication of a problem, and therefore a higher low saturation threshold may be desirable. Similarly, a patient diagnosed with coronary artery disease (CAD) or congestive heart failure (CHF) may have a diminished ability to increase oxygen delivery by increasing blood flow. As a result, a low saturation threshold of 88-90 percent may be set for such a patient.

Furthermore, algorithms utilized to calculate the patient's physiological parameters may be more or less useful based on the patient's characteristics. For example, a young child is more likely to make frequent movements than an elderly patient. Accordingly, when patient characteristics indicate that a patient is below a certain age, a feature may be enabled which reduces false alarms based on minor, short-term deviations such as those caused by motion artifacts. For example, oxygen saturation alarms may be delayed until the time-integral between the calculated saturation value and the alarm threshold exceeds a certain value. The SatSeconds™ alarm management technology, such as that available in the Model N600x monitor available from Nellcor Puritan Bennett LLC, may provide such an alarm reduction feature. Accordingly, for patients below a predetermined age (i.e., 3 years), such an integral-based alarm management technique may be automatically enabled, or the threshold value for this alarm integral may be set to a higher value than for older patients.

Additionally, certain patient characteristics may be indicative of a high risk of sleep apnea. For example, obesity, high neck circumference, other anatomical anomalies of the upper airway, or high doses of pain medications that are known to depress intrinsic muscle tone, such as opoids, may increase a patient's risk of experiencing sleep apnea. Accordingly, a feature to detect the occurrence of desaturation patterns indicative of ventilation instability may be enabled to alert the caregiver to frequent desaturation events.

While the subject of this disclosure may be susceptible to various modifications and alternative forms, specific embodiments have been shown by way of example in the drawings and have been described in detail herein. However, it should be understood that this disclosure is not intended to be limited to the particular forms disclosed. Rather, the invention is to cover all modifications, equivalents, and alternatives falling within the spirit and scope of the invention as defined by the following appended claims.

What is claimed is:

1. A method for setting alarm thresholds, comprising:
receiving patient characteristics onto a memory device; and
using a processor to determine alarm thresholds based on the patient characteristics.

2. The method of claim 1, wherein the patient characteristics comprise age, weight, height, diagnosis, medications, treatments, or a combination thereof.

3. The method of claim 1, wherein receiving comprises receiving the patient characteristics from a user input interface.

4. The method of claim 1, wherein receiving comprises receiving the patient characteristics from a sensor or a radio frequency identification tag.

5. The method of claim 1, wherein receiving comprises receiving the patient characteristics from a network.

6. The method of claim 1, wherein using a process to determine comprises calculating the alarm thresholds using one or more algorithms.

7. The method of claim 1, wherein using a process to determine comprises looking up the alarm thresholds in one or more look-up tables.

8. A patient monitoring system, comprising:
an input device configured to receive information about a patient;
a first memory device configured to store specifications for determining alarm thresholds based on the received information;
a processor configured to determine the alarm thresholds based on the received information and using the stored specifications; and
a second memory device configured to store the determined alarm thresholds for comparison to physiological parameters of the patient.

9. The patient monitoring system of claim 8, comprising a sensor configured to gather data correlating to the physiological parameters.

10. The patient monitoring system of claim 9, wherein the sensor comprises the first memory device.

11. The patient monitoring system of claim 97 wherein:
the first memory device is configured to store algorithms for calculating the physiological parameters; and
the processor is configured to calculate the physiological parameters based on the gathered data and the stored algorithms.

12. The patient monitoring system of claim 8, wherein the processor is configured to compare the physiological parameters to the alarm thresholds and send a signal to an alarm device if at least one of the physiological parameters exceeds the alarm thresholds.

13. A method, comprising:
receiving one or more signals from a sensor, the one or more signals corresponding to absorption of light in a patient's tissue;
calculating one or more physiological parameters of the patient based on the one or more signals;
receiving one or more patient characteristics;
determining one or more threshold ranges of the physiological parameters based on the one or more patient characteristics; and
providing a notification if at least one of the calculated physiological parameters is outside a corresponding threshold range of the one or more threshold ranges.

14. The method of claim 13, wherein the physiological parameters comprise heart rate, blood oxygen saturation, or a combination thereof.

15. The method of claim 13, wherein the patient characteristics comprise age, weight, height, diagnosis, medications, treatments, or a combination thereof.

16. The method of claim 13, wherein receiving the one or more patient characteristics comprises receiving the patient characteristics from a user input interface, a sensor, a radio frequency identification tag, a network, or a combination thereof.
